(19) United States
(12) Patent Application Publication
(10) Pub. No.: US 2020/0330261 A1
(43) Pub. Date: Oct. 22, 2020

(54) SYSTEM AND METHOD FOR TREATING BRUXISM

(71) Applicant: John Joseph Anderson Jones, Sherman, TX (US)

(72) Inventor: John Joseph Anderson Jones, Sherman, TX (US)

(21) Appl. No.: 16/851,025

(22) Filed: Apr. 16, 2020

Related U.S. Application Data
(60) Provisional application No. 62/836,123, filed on Apr. 19, 2019.

(51) Int. Cl. A61F 5/56; A61N 1/05; A61N 1/36; A61N 1/372

(52) U.S. Cl. CPC A61F 5/566; A61N 1/0548; A61N 1/36139; A61N 1/37252; A61F 2005/563; A61N 1/36178; A61N 1/36192; A61N 1/36196; A61N 1/36167; A61N 1/37217

(57) ABSTRACT

A bruxism prevention system has a mouthpiece comprising a pressure sensor, an electrode, and an electrical stimulation generator. The bruxism prevention system also includes a controller configured to manage application of electrical stimulation to a user via the electrode in response to a pressure sensed by the pressure sensor. A method of preventing bruxism includes disposing an electrode within a user's mouth, disposing a pressure sensor between the user's upper jaw and the user's lower jaw, disposing an electrical stimulation generator within the user's mouth, sensing a predetermined pressure, and applying electrical stimulation to the user's mouth via the electrode in response to the sensed predetermined pressure.

### CROSS-REFERENCE TO RELATED APPLICATIONS

This patent application claims the benefit of the filing date of U.S. Provisional Patent Application Ser. No. 62/836,123 filed on 19 Apr. 2019 and entitled “SYSTEM AND METHOD FOR TREATING BRUXISM,” the entire content of which is hereby expressly incorporated by reference.

### STATEMENT REGARDING FEDERALLY SPONSORED RESEARCH OR DEVELOPMENT

Not applicable.

### BACKGROUND

Bruxism is a condition defined as a condition where a person clenches or grinds their teeth uncontrollably and unconsciously. Emotional or psychological stimuli such as stress and anxiety or physical factors such as crooked teeth, missing teeth, or abnormal bite can contribute to the prevalence of bruxism. The frequent and repetitive grinding forces generated by bruxism can cause fracturing, loosening, and loss of teeth. Repairing physical damage to the teeth includes the use of implants, root canals, and crowns, and may even require full dentures when repair procedures fail. Along with mechanical damage to teeth, headaches and osteoarthritis of the mandibular joint are possible symptoms of bruxism. Research also points to bruxism as a major cause of temporomandibular joint disorder (TMJ) and other temporomandibular disorders (TMDs).

Current methods for alleviation or cessation of bruxism are largely based on custom-fitted mouthpieces. A well-known variant of the custom-fitted mouthpieces is known as an intra-oral occlusal splint, colloquially referred to as a night guard. This splint is formed to fit the shape of a patient's teeth and serves to prevent damage that would otherwise be incurred by grinding. Unfortunately, the splint cannot relieve the effects of bruxism on the mandibular joint or cranial region. By design, the splints can only treat and prevent some symptoms, rather than the underlying cause, the clenching and grinding actions. Other drawbacks of the splint include patients grinding through the splint over time (requiring replacement of the splint), difficulty in maintaining cleanliness of the splint, and obstruction of speech caused by the splint while worn.

Other solutions have been attempted in an effort to provide some method of rectifying the cause of the bruxism itself rather than simply masking the symptoms. One such solution includes a mouthpiece that contains a source of electrical power and an electrode capable of making contact with the interior of the user's mouth. When the user closes their mouth and upper and lower components of the mouthpiece make contact, a circuit is formed which initiates an electrical impulse emitted from the electrode. While utilizing electricity to stimulate corrective actions on the part of the patient, this invention does not include the option of recharging its power source, a conventional battery. Assuming a nominal 150-hour battery life and considering a 7-hour sleep cycle, such a battery may only last 21.4 days before requiring replacement.

Another solution uses piezoelectric elements embedded in a mouthpiece to generate a small electrical current when a user activates them through excessive bite force. This current activates a miniaturized radio transmitter that is also embedded in the mouthpiece. The signal from this transmitter travels to a receiver that amplifies the gain and creates an audible tone to alert the user to relax their j aw. A drawback of this system is that the user must be awake and within range of the receiver in order for this method to provide the necessary corrective feedback.

### SUMMARY

This disclosure divulges an improved system and method of treating bruxism. The system comprises a mouthpiece, at least one permanent battery, an electrical system that allows for inductive charging, at least one electrode for emitting treatment impulses, at least one piezoelectric bite pressure transducer, a miniaturized wireless transceiver, and an integrated circuit pattern controller. The system is operable to generate an electrical impulse in response to activation of the piezoelectric pressure transducer. The piezoelectric pressure transducer is generally activated in response to a bite pressure exerted on the piezoelectric pressure transducer exceeding a predetermined pressure threshold. The electrical impulse is transmitted to the inside of the user's cheek through the electrode and causes an immediate reduction in mandibular muscle tension and resultant reduction of pressure applied to the mouthpiece. Power is supplied via a permanently mounted rechargeable battery encased within the mouthpiece. The battery is configured to be rechargeable using an inductive charging system, thereby eliminating the need for charge ports which may be subject to fluid penetration and corrosion.

Operation of the system is achieved using integrated circuit logic that stores, activates and learns various activation profiles. The integrated circuit communicates to external devices via a miniaturized wireless transceiver utilizing Bluetooth, Wi-Fi, or other suitable wireless data transmission technologies. The external device may comprise a smartphone application or a standalone remote control. The smartphone application is configured to analyze impulse activation patterns via adaptive learning algorithms and creates profiles that best suit the specific user. In some embodiments, a manual mode is available for users who prefer increased control over activation threshold pressure, shock intensity, and diurnal variations.

### BRIEF DESCRIPTION OF THE DRAWINGS

FIG. 1 is a top view of a bruxism treatment system (BTS) according to this disclosure.

FIG. 2 is a front view of the BTS of FIG. 1.

FIG. 3 is a side view of the BTS of FIG. 1.

FIG. 4 is a simplified schematic of a portion of the BTS of FIG. 1.

FIG. 5 is a view of an interactive interface for use with the BTS of FIG. 1.

FIG. 6 is a view of an information display for use with the BTS of FIG. 1.

FIG. 7 is another view of an information display for use with the BTS of FIG. 1.

FIG. 8 is a top view of an alternative embodiment of a BTS mouthpiece.

FIG. 9 is a side view of the BTS of FIG. 8.

FIG. 10 is a schematic representation of a simple switched circuit implementation for controlling application of stimulation applied by a BTS.

FIG. 11 is a schematic representation of a piezoelectric switched circuit implementation for controlling application of stimulation applied by a BTS.

FIG. 12 is a flowchart of a method of operating a BTS according to a sleep mode.

FIG. 13 is a flowchart of a method of operating a BTS according to a manual or awake mode.

FIG. 14 is a graphical representation of a burst pattern.

FIG. 15 is a graphical representation of a continuous pattern.

FIG. 16 is a graphical representation of an amplitude modulated pattern.

FIG. 17 is a graphical representation of a low-frequency pattern.

FIG. 18 is a graphical representation of a high frequency pattern.

FIG. 19 is a graphical representation of a high amplitude pattern.

FIG. 20 is a graphical representation of a low amplitude pattern.

FIG. 21 is a graphical representation of a short duration pattern.

FIG. 22 is a graphical representation of a long duration pattern.

### DETAILED DESCRIPTION

In this disclosure, reference may be made to the spatial relationships between various components and to the spatial orientation of various aspects of components as the devices are depicted in the attached drawings. However, as will be recognized by those skilled in the art after a complete reading of this disclosure, the devices, members, apparatuses, etc. described herein may be positioned in any desired orientation. Thus, the use of terms such as “above,” “below,” “upper,” “lower,” or other like terms to describe a spatial relationship between various components or to describe the spatial orientation of aspects of such components should be understood to describe a relative relationship between the components or a spatial orientation of aspects of such components, respectively, as the device described herein may be oriented in any desired direction.

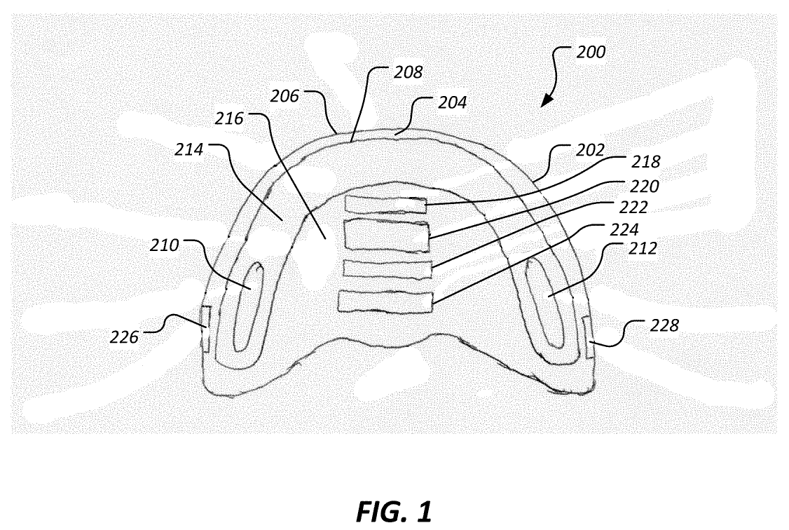

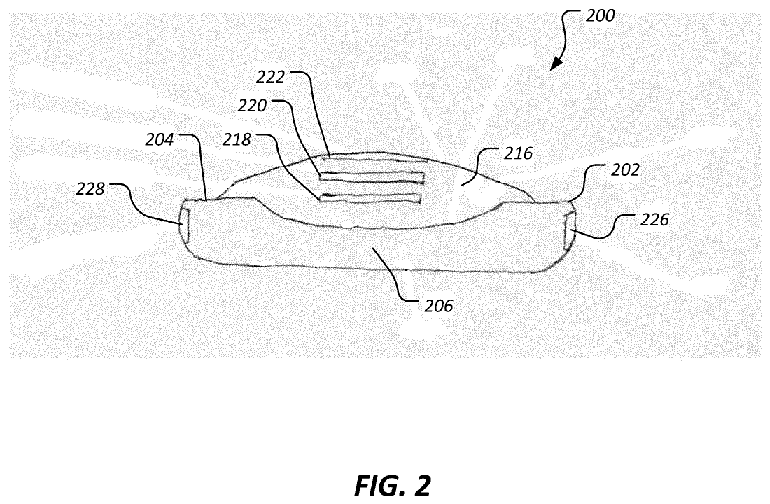

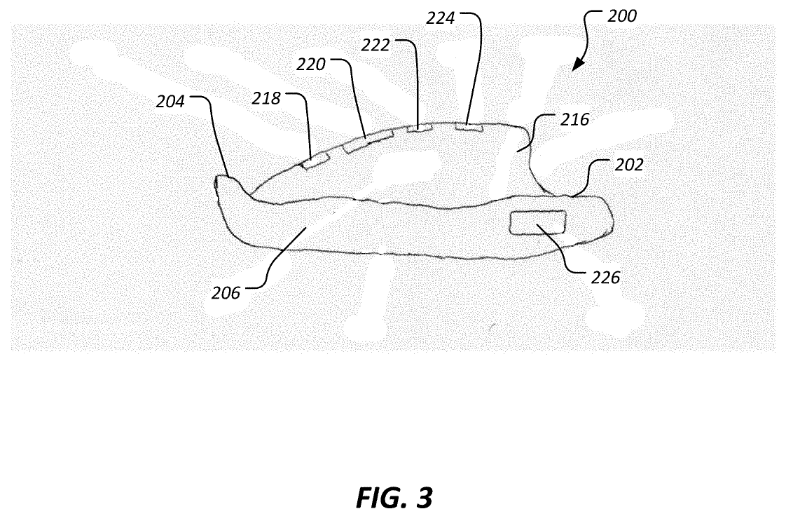

With reference to FIG. 1-3, a BTS 200 is shown. BTS 200 comprises a mouthpiece 202 having a curved exterior tooth wall 204. The exterior tooth wall 204 comprises an outer surface 206 that generally faces away from adjacent teeth and an inner surface 208 that may generally contact adjacent teeth. The mouthpiece 202 is generally configured for being worn and carried by a user's upper teeth. However, in alternative embodiments, a BTS substantially similar to BTS 200 can be provided and configured to be worn on and carried by a user's lower teeth. Regardless of whether the BTS is designed for being worn by upper teeth or lower teeth, a bite pad 214 attached to the exterior tooth wall 204 is provided for selective engagement by opposing upper and lower teeth. The profiles and contours of the exterior tooth wall 204 and the bite pad 214 can be generic in construction or can be customized by dental professionals to fit the specific physical features of a user. The mouthpiece 202 can be constructed of various materials compatible with placement in a human mouth and sufficiently resistant to high biting pressures without causing damage to a user's teeth. In some embodiments, the mouthpiece 202 is configured to fit the upper teeth to simplify stability and increase user comfort, although as noted above, construction of a mouthpiece for the bottom teeth is also possible.

The mouthpiece 202 further comprises a curved roof plate 216 that extends from the bite pad 214 in a direction configured to locate the roof plate 216 generally between a user's tongue and the roof of the user's mouth when worn. An integrated circuit controller 220 containing the logic for the operation of the BTS 200 is disposed within the roof plate 216. The integrated circuit controller 220 is also configured to utilize a memory for storage of activation and stimulation events. The integrated circuit controller 220 is further configured to control the above-mentioned inductive charging of a battery 224. A wireless transceiver 222, also disposed within the roof plate 216, is used for transmitting and receiving data when communicating with external controllers or monitors such as, but not limited to, the above-mentioned smartphone application and/or dedicated remote controller.

Piezoelectric bite pressure transducers 210, 212 are shown in FIG. 1. The piezoelectric bite pressure transducers 210, 212 are configured to selectively enable signaling to the integrated circuit controller 220 when a bite pressure exceeds the above-described predetermined pressure threshold. The signaling and/or other outputs provided by or enabled by the piezoelectric bite pressure transducers 210, 212 can be linearly or exponentially correlated to the amplitude of bite pressure applied to the piezoelectric bite pressure transducers 210, 212.

Two electrodes 226, 228 are generally disposed at least partially inset within the exterior tooth wall 204 and either flush with or extending outwardly beyond the outer surface 206 The electrodes 226, 228 are disposed on opposing sides of the mouthpiece 202 so that both cheeks can be contacted. When the electrodes 226, 228 are in contact with a user's cheeks, the above-describe electrical impulses, shocks, and/or stimulations can be administered to the cheeks in response to the above-described bite pressure exceeding a predetermined pressure threshold. Contact with the inner lining of a user's cheeks and thus transmit electrical impulses in response to bite pressure. With this in mind, electrodes 226, 228 are constructed of an electrically conductive material and shaped to concentrate an energy discharge to an adequate level for stimulation.

A charge plate 218 for the inductive charging system is provided and disposed within the roof plate 216. In some embodiments, the charge plate 218 can be shielded to prevent unwanted bio-electrical interference to the user. Placing the charge plate 218 in sufficient physical proximity to an inductive charging device, a transfer of electrical energy will occur that recharges the battery 224 located within the roof plate 216. The battery 224 is embedded in the mouthpiece 202 and is not intended for replacement at any point during the lifespan of the BTS 200.

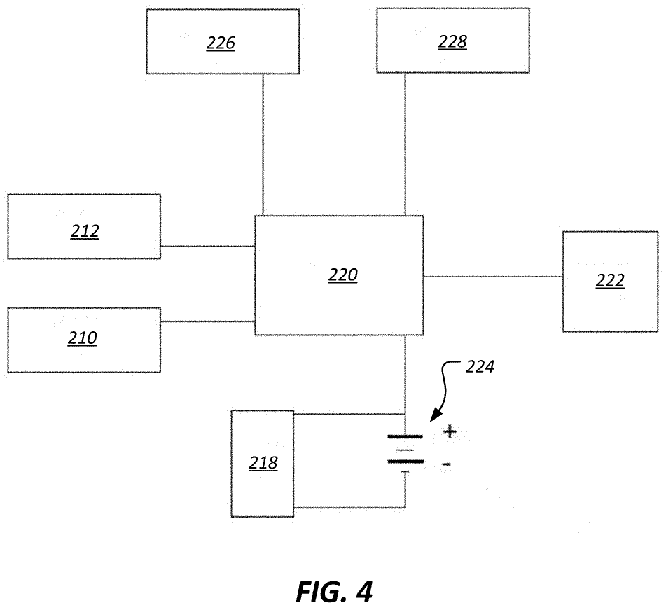

FIG. 4 shows a simplified schematic diagram of a portion of the BTS 200. A user that bites down with sufficient pressure to exceed the predetermined pressure threshold activates one or both piezoelectric bite pressure transducers 210, 212, triggering an electrical signal that travels to the integrated circuit controller 220. The integrated circuit controller 220, based on predetermined response curves, commands a discharge of electricity from the battery 224 to the electrodes 226, 228. The integrated circuit controller 220 can cause storage of information to a memory device regarding the pressure exerted, discharge intensity, time, date, duration of pressure exerted, and duration of discharge emitted. This information can be saved internally to the memory but can also be transmitted to an external device 300 for study and trend monitoring. Sending and receiving data is accomplished by the wireless transceiver 222 that facilitates all communication between the BTS 200 and the external device 300.

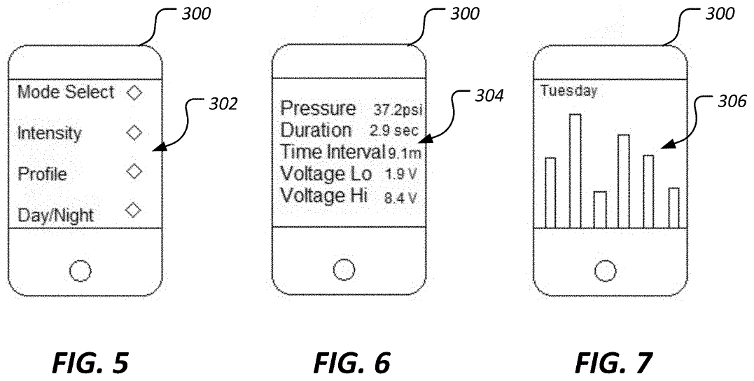

FIG. 5 shows an example interactive interface 302 of an external device 300 where the external device 300 comprises a smartphone. The external device 300 is configured to provide an interactive interface 302 that allows selection of a manual mode of operation or an automatic mode of operation of the BTS 200, thereby allowing user to fine tune their experience. In some embodiments, the interactive display can allow a user to control stimulation intensity, threshold pressure, day versus night variation, and automatic data uploading. For example, if a user selects “Sleep Mode”, the day/night variance function can reduce the intensity and duration of a stimulation to prevent waking the user while relaxing the mandibular muscles to relieve bite pressure. The integrated circuit controller 220 uses an adaptive learning algorithm to determine the best settings based on historical data, however it can also utilize information from an external device 300, such as the above-mentioned smartphone or a wearable fitness tracker. In cases where a wearable fitness tracker comprising heartrate sensors is utilized, a reported heartrate could be used to determine when a user is asleep. The information provided to the external device 300 can be evaluated and manipulated to generate revised control algorithms for delivery to the BTS 200 via the wireless transceiver 222.

FIGS. 6 and 7 shows example information displays 304, 306, respectively, of the external device 300. Users can operate the external device 300 to switch between information displays 304, 306. Using information displays 304, 306, a user can see specific information about their bruxism related habits and the corrective actions taken by the BTS 200. In some embodiments, this data can be automatically uploaded to an external server for long term trend monitoring. The data can be displayed numerically as in the case of information display 304 or graphically as in the case of information display 306, depending on the preference of the user or individual monitoring the usage history of the BTS 200.

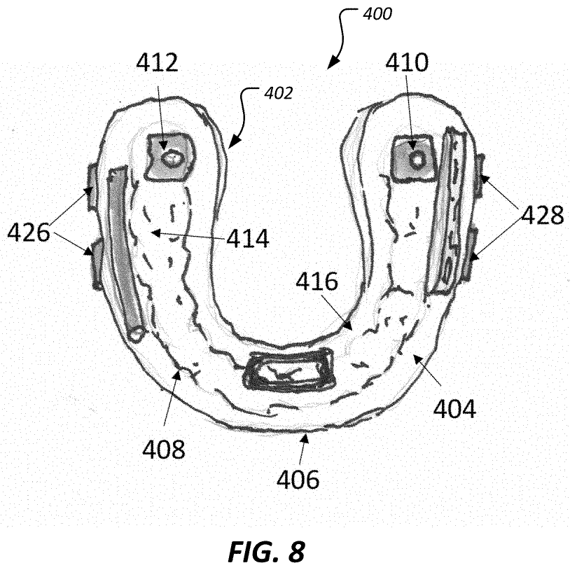

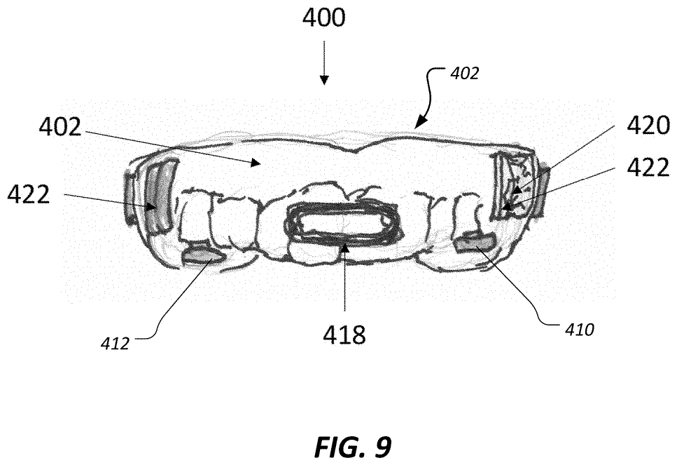

With reference to FIGS. 8-9, an alternative embodiment of a BTS 400 is shown. BTS 400 comprises a mouthpiece 402 having a curved exterior tooth wall 404. The exterior tooth wall 404 comprises an outer surface 406 that generally faces away from adjacent teeth and an inner surface 408 that may generally contact adjacent teeth. The mouthpiece 402 is generally configured for being worn and carried by a user's upper teeth. However, in alternative embodiments, a BTS substantially similar to BTS 400 can be provided and configured to be worn on and carried by a user's lower teeth. Regardless of whether the BTS is designed for being worn by upper teeth or lower teeth, a bite pad 414 attached to the exterior tooth wall 404 is provided for selective engagement by opposing upper and lower teeth. The profiles and contours of the exterior tooth wall 404 and the bite pad 414 can be generic in construction or can be customized by dental professionals to fit the specific physical features of a user. The mouthpiece 402 can be constructed of various materials compatible with placement in a human mouth and sufficiently resistant to high biting pressures without causing damage to a user's teeth. In some embodiments, the mouthpiece 402 is configured to fit the upper teeth to simplify stability and increase user comfort, although as noted above, construction of a mouthpiece for the bottom teeth is also possible.

The mouthpiece 402 further comprises a curved interior tooth wall 416 that extends from the bite pad 414 in a direction configured to locate the curved interior tooth wall 416. An integrated circuit controller 420 containing the logic for the operation of the BTS 400 is disposed within the curved exterior tooth wall 404. The integrated circuit controller 420 may also be configured to utilize a memory for storage of activation and stimulation events. The integrated circuit controller 420 is further configured to control the above-mentioned inductive charging of a battery 424. A wireless transceiver 422, also disposed within the curved exterior tooth wall 404, is used for transmitting and receiving data when communicating with external controllers or monitors such as, but not limited to, the above-mentioned smartphone application and/or dedicated remote controller. Piezoelectric bite pressure switches, actuator buttons or Ohm impedance switches 410, 412 are shown in FIG. 9. The piezoelectric bite pressure switches, actuator button or Ohm impedance switches 410, 412 are configured to selectively enable signaling to the integrated circuit controller 420 when a bite pressure exceeds the above-described predetermined pressure threshold. The signaling and/or other outputs provided by or enabled by the piezoelectric bite pressure switches, actuator buttons or Ohm impedance switches 410, 412 can be linearly or exponentially correlated to the amplitude of bite pressure applied to the piezoelectric bite pressure switches, actuator buttons or Ohm impedance switches 410, 412. While bite pressure switches 410, 412 are shown as separate components, in alternative embodiments, a single bite pressure transducer can be provided that is disposed in substantially the same locations as the bite pressure transducers 410, 412, as well as at other locations within the mouthpiece 402. Two electrodes 426, 428 are generally disposed at least partially inset within the exterior tooth wall 404 and either flush with or extending outwardly beyond the outer surface 406. The electrodes 426, 428 may be disposed on opposing sides of the mouthpiece 402 so that both cheeks can be contacted or may only be on one side. When the electrodes 426, 428 are in contact with a user's cheeks, the above describe electrical impulses, shocks, and/or stimulations can be administered to the cheeks in response to the above-described bite pressure exceeding a predetermined pressure threshold. Contact with the inner lining of a user's cheeks and thus transmit electrical impulses in response to bite pressure. With this in mind, electrodes 426, 428 are constructed of an electrically conductive material and shaped to concentrate an energy discharge to an adequate level for stimulation. A charge coil 418 for the inductive charging system is provided and disposed within the curved interior tooth wall 416. In some embodiments, the charge coil 418 can be shielded to prevent unwanted bio-electrical interference to the user. Placing the charge coil 418 in sufficient physical proximity to an inductive charging device, a transfer of electrical energy will occur that recharges the battery 424 located within the curved interior tooth wall 416. The battery 424 is embedded in the mouthpiece 402 and is not intended for replacement at any point during the lifespan of the BTS 400.

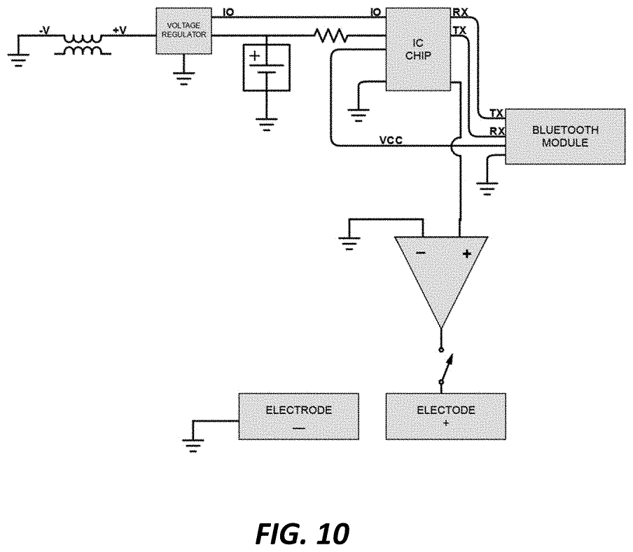

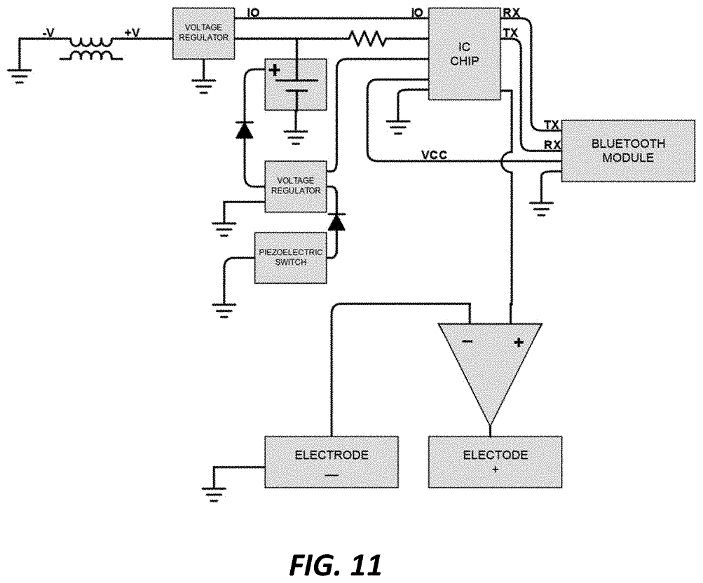

FIG. 10 shows a simplified schematic diagram of a conventional switched implementation of a portion of an embodiment of the BTS 400. FIG. 11 shows a simplified schematic diagram of a piezoelectric switched embodiment of a portion of an alternative embodiment of the BTS 400. A user that bites down with sufficient pressure to exceed the predetermined pressure threshold activates one or multiple of the piezoelectric bite pressure switches, actuator buttons or Ohm impedance switches 410, 412, triggering an electrical signal that travels to the integrated circuit controller 420. The integrated circuit controller 420, based on predetermined response curves, commands a discharge of electricity from the battery 424 to the electrodes 426, 428. The integrated circuit controller 420 can cause storage of information to a memory device regarding the pressure exerted, discharge intensity, time, date, duration of pressure exerted, and duration of discharge emitted. This information can be saved internally to the memory but can also be transmitted to an external device (such as external device 300) for study and trend monitoring. Sending and receiving data is accomplished by the wireless transceiver 422 that facilitates communication between the BTS 400 and the external device.

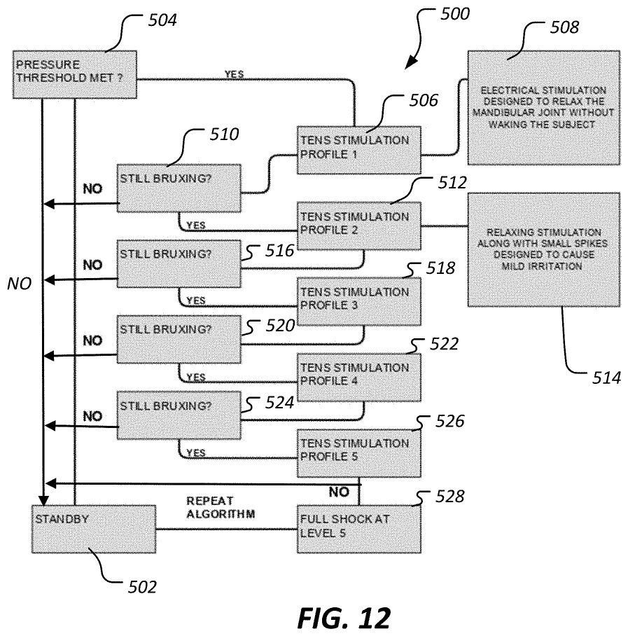

Referring now to FIG. 12, a simplified flow chart of a method 500 of operation of a BTS is shown. As previously mentioned, a BTS according to one or more of the embodiments disclosed herein can be operated in a variety of modes, such as, but not limited to, a night mode of operation. The night mode of operation is intended to detect bruxing through one or more of the above-described pressure sensors and in response to the detected bruxing, apply stimulation through the electrodes preferably in a manner that does not rouse or awaken the user of the BTS. Because users inherently comprise different pain or stimulation sensing thresholds, the BTSs disclosed herein can be configured to gradually cycle through a variety of stimulation profiles with the goal of utilizing a stimulation profile with at least one of a lowest stimulation amplitude and/or a lowest perceived stimulation sensation that is effective to interrupt the bruxing. One example implementation of a sleep mode algorithm for interrupting or ceasing bruxing is shown in FIG. 12 as method 500.

Method 500 can begin at block 502 wherein the BTS is in a standby mode during which no stimulation is being administered. The method 500 can progress from block 502 to block 504 at which the BTS is configured to poll a status of a pressure switch to determine whether a user is bruxing. There is not bruxing, the method 500 may return to block 502 and remain in the standby mode for a predetermined amount of time. However, if at block 504 the user is bruxing (applying a sufficient amount of pressure to one or more pressure sensors to activate the sensor) the method 500 may progress to block 506. At block 506, the method 500 may apply a transcutaneous electrical nerve stimulation (TENS) according to a first profile. The first profile may comprise an electrical stimulation designed to relax the mandibular joint without waking the user as shown at block 508. After the method 500 applies the first profile of stimulation at block 506, the method 500 may progress to block 510 where the method 500 determines whether the user is continuing bruxing after application of the first profile of stimulation. If the user is no longer bruxing, the method 500 has been successful in discontinuing or interrupting the bruxing and may return to the standby mode at block 502 For a predetermined period of time before again progressing to block 504. However, if at block 510 the user is determined to continue bruxing, the method 500 may progress to block 512. At block 512 the method 500 may apply a TENS according to a second profile. The second profile may comprise an electrical stimulation intended to relax the mandibular joint but also provide small spikes in amplitude of stimulation that are selected to cause only mild irritation to the user. In this embodiment, the maximum amplitude of the second profile is greater than the maximum amplitude of the first profile. After applying the stimulation at block 512, the method 500 may progress to block 516 to again determine whether the user is bruxing. If the user is no longer bruxing, the method 500 may progress back to block 502. However, if the user is still bruxing, the method 500 may progress to block 518 and apply a TENS according to a third profile. In some embodiments a maximum amplitude and/or duration of the stimulation applied at block 518 may be greater than the maximum amplitude and/or duration of the stimulation applied at block 512. After applying the stimulation at block 518, the method 500 may progress to block 522 once again determine whether the user is bruxing. If the user is no longer bruxing, the method 500 may progress back to block 502. However, if the user is still bruxing, the method 500 may progress to block 522 and apply a TENS according to fourth profile. The fourth profile stimulation may comprise a greater amplitude and/or duration of stimulation as compared to the third profile. After applying the stimulation at block 522, the method 500 may progress to block 524 to determine if the user is still bruxing. If the user is no longer bruxing, the method 500 may progress back to block 502. However, if the user is still bruxing, the method 500 may progress to block 526 and apply a TENS according to a fifth profile. In some embodiments, the stimulation of the fifth profile may comprise a greater amplitude and/or duration of stimulation as compared to the fourth profile. After applying the stimulation at block 526, the method 500 may progress back to block 502 if the user has stopped bruxing. However, if the application of the fifth profile stimulation has not interrupted or stopped the bruxing, the method 500 may progress to block 528 at which a maximum amplitude of stimulation is administered. After administering the stimulation at block 528, the method may return to block 502. In some embodiments, the method 500 may further comprise steps for recording whether the application of certain stimulation profiles was successful for a particular user and utilize that information to adjust and amplitude and/or duration of stimulation at one or more of the blocks 506, 512, 518, 522, 526, 528. Accordingly, subsequent progression through the flowchart of method 500 may accomplish cessation of bruxism at profile steps occurring prior to the stimulation at block 528.

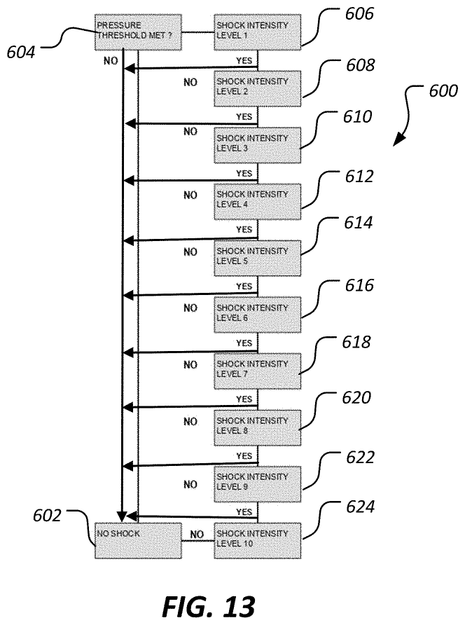

Referring now to FIG. 13, a simplified flow chart of a method 600 of operation of a BTS is shown. As previously mentioned, a BTS according to one or more of the embodiments disclosed herein can be operated in a variety of modes, such as, but not limited to, a day mode of operation. The day or manual progressive amplitude mode of operation is intended to detect bruxing through one or more of the above-described pressure sensors and in response to the detected bruxing, apply stimulation through the electrodes preferably in a manner that a alerts a user who is awake to the fact that they are bruxing so that the user can consciously discontinue the bruxing. Accordingly the method 600 is configured to operate without the same concern of waking the user and instead is designed to be implemented as an operant conditioning methodology whereby the user can form better habits with regard to awareness of bruxing in response to the discomfort received by progressively uncomfortable stimulations.

Method 600 can begin at block 602 where there is no stimulation being applied to the electrodes of the BTS. Method 600 can progress to block 604 where one or more of the pressure sensors can be polled to determine whether the user is bruxing as a function of determining whether a predetermined pressure threshold has been met or sentenced by the pressure sensors. If the user is determined to not be bruxing at block 604, the method 600 can return to block 602. After predetermined amount of time, the method may again progress from block 602 to block 604. However, if at block 604 the method 600 determines that the user is bruxing, the method may progress to block 606 where a TENS is applied at a first amplitude level. If after application of the stimulation at block 606 stops the bruxing, the method 600 may progress back to block 602. However, if the user continues bruxing after application of the stimulation at block 606, the method 600 may progress to block 608. At block 608, the method 600 may apply a stimulation of greater amplitude and/or duration as compared to the stimulation applied at block 606. If after application of the stimulation at block 608 stops the bruxing, the method 600 may progress back to block 602. However, if the user continues bruxing after application of the stimulation at block 608, the method 600 may progress to block 610. At block 610, the method 600 may apply a stimulation of greater amplitude and/or duration as compared to the stimulation applied at block 608. If after application of the stimulation at block 610 stops the bruxing, the method 600 may progress back to block 602. However, if the user continues bruxing after application of the stimulation at block 610, the method 600 may progress to block 612. At block 612, the method 600 may apply a stimulation of greater amplitude and/or duration as compared to the stimulation applied at block 610. If after application of the stimulation at block 612 stops the bruxing, the method 600 may progress back to block 602. However, if the user continues bruxing after application of the stimulation at block 612, the method 600 may progress to block 614. At block 614, the method 600 may apply a stimulation of greater amplitude and/or duration as compared to the stimulation applied at block 612. If after application of the stimulation at block 614 stops the bruxing, the method 600 may progress back to block 602. However, if the user continues bruxing after application of the stimulation at block 614, the method 600 may progress to block 616. At block 616, the method 600 may apply a stimulation of greater amplitude and/or duration as compared to the stimulation applied at block 614. If after application of the stimulation at block 616 stops the bruxing, the method 600 may progress back to block 602. However, if the user continues bruxing after application of the stimulation at block 616, the method 600 may progress to block 618. At block 618, the method 600 may apply a stimulation of greater amplitude and/or duration as compared to the stimulation applied at block 616. If after application of the stimulation at block 618 stops the bruxing, the method 600 may progress back to block 602. However, if the user continues bruxing after application of the stimulation at block 618, the method 600 may progress to block 620. At block 620, the method 600 may apply a stimulation of greater amplitude and/or duration as compared to the stimulation applied at block 618. If after application of the stimulation at block 620 stops the bruxing, the method 600 may progress back to block 602. However, if the user continues bruxing after application of the stimulation at block 620, the method 600 may progress to block 622. At block 622, the method 600 may apply a stimulation of greater amplitude and/or duration as compared to the stimulation applied at block 620. If after application of the stimulation at block 622 stops the bruxing, the method 600 may progress back to block 602. However, if the user continues bruxing after application of the stimulation at block 622, the method 600 may progress to block 624. At block 624, the method 600 may apply a stimulation of greater amplitude and/or duration as compared to the stimulation applied at block 622.

In some embodiments, the method 600 may further comprise steps for recording whether the application of certain stimulation levels were successful for a particular user and utilize that information to adjust and amplitude and/or duration of stimulation at one or more of the blocks 606, 608, 610, 612, 614, 616, 618, 620, 622, and 624. Accordingly, subsequent progression through the flowchart of method 600 may accomplish cessation of bruxism at profile steps occurring prior to the stimulation at block 624. In other embodiments, the method 600 may be configured to utilize a determined successful level of stimulation intensity to bracket a smaller range of stimulation so that initial stimulations start below the successful stimulation intensity but higher than the intensity initially applied at block 606. In this way the method 600 could become adaptive so that the same initially determined successful intensity level is not the starting point, but rather is more quickly approached while still offering the user a lower stimulation intensity. In some cases, a user may be conditioned to be more aware of lower stimulation intensities so that over time the stimulations that successfully stop the bruxism are progressively lower. Ideally, the successful stimulation intensities would become very low and/or the user may be conditioned so that continued use of the BTS is not necessary because the user has formed new awareness and habits regarding bruxism.

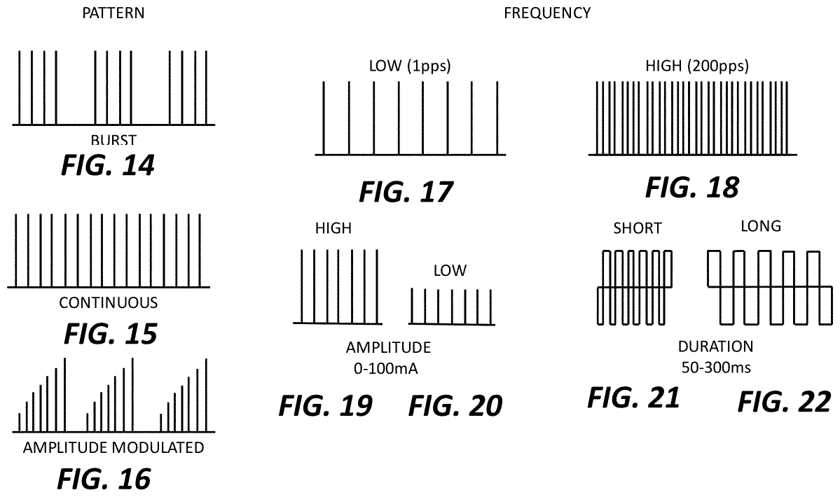

Referring now to FIG. 14, a graphical representation of a burst type pattern of stimulation is shown where stimulation is repeated in tight time-spaced groups with similar amplitude.

Referring now to FIG. 15, a graphical representation of a continuous type pattern of stimulation is shown where stimulation is repeated in equally time-spaced application with similar amplitude.

Referring now to FIG. 16, a graphical representation of an amplitude modulated type pattern of stimulation is shown where an amplitude of stimulation is gradually increased within time-spaced groups and with each group restarting at a set lower amplitude.

Referring now to FIG. 17, a graphical representation of a low-frequency application of stimulation is accomplished by applying stimulation of a same amplitude relatively infrequently, such as one pulse per second.

Referring now to FIG. 18, a graphical representation of a high-frequency application of stimulation is accomplished by applying stimulation of a same amplitude relatively more frequently as compared to the profile of FIG. 17, such as 200 pulses per second.

Referring now to FIG. 19, a graphical representation of a high amplitude stimulation application at regular intervals.

Referring now to FIG. 20, a graphical representation of a relatively lower amplitude stimulation application (as compared to the profile of FIG. 19) at regular intervals.

Referring now to FIG. 21, a graphical representation of a short duration type pattern of stimulation is shown where stimulation is applied for a relatively short time such as 50 milliseconds at a time and in evenly spaced intervals.

Referring now to FIG. 22, a graphical representation of a relatively longer duration type pattern of stimulation (as compared to the profile of FIG. 21) is shown where stimulation is applied for a relatively longer time such as 300 milliseconds at a time and in evenly spaced intervals.

Alternative embodiments that result from combining, integrating, and/or omitting features of the embodiment(s) are also within the scope of this disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). For example, whenever a numerical range with a lower limit, Rₗ, and an upper limit, Rᵤ, is disclosed, any number falling within the range is specifically disclosed. In particular, the following numbers within the range are specifically disclosed: R=Rₗ+k*(Rᵤ−Rₗ), wherein k is a variable ranging from 1 percent to 100 percent with a 1 percent increment, i.e., k is 1 percent, 2 percent, 3 percent, 4 percent, 5 percent, . . . 50 percent, 51 percent, 52 percent, . . . , 95 percent, 96 percent, 95 percent, 98 percent, 99 percent, or 100 percent. Moreover, any numerical range defined by two R numbers as defined in the above is also specifically disclosed. Use of the term “optionally” with respect to any element of a claim means that the element is required, or alternatively, the element is not required, both alternatives being within the scope of the claim. Use of broader terms such as comprises, includes, and having should be understood to provide support for narrower terms such as consisting of, consisting essentially of, and comprised substantially of. Accordingly, the scope of protection is not limited by the description set out above but is defined by the claims that follow, that scope including all equivalents of the subject matter of the claims. Each and every claim is incorporated as further disclosure into the specification and the claims are embodiment(s) of the present invention. Also, the phrases “at least one of A, B, and C” and “A and/or B and/or C” should each be interpreted to include only A, only B, only C, or any combination of A, B, and C.

What is claimed is:

1. A bruxism prevention system, comprising:
a mouthpiece comprising a pressure sensor, an electrode, and an electrical stimulation generator; and
a controller configured to manage application of electrical stimulation generated by the electrical stimulator.

2. The system of claim 1, wherein the pressure sensor is a pressure switch.

3. The system of claim 1, wherein the pressure sensor is embedded within the mouthpiece.

4. The system of claim 1, wherein the electrode is configured to contact the inner cheek of a user.

5. The system of claim 1, wherein the controller is configured to selectively apply electrical stimulation according to an algorithm.

6. The system of claim 5, wherein the controller is configured to successively progress through a plurality of different stimulation profiles until a predetermined change in pressure is sensed by the pressure sensor.

7. The system of claim 5, wherein the controller is configured to wirelessly communicate with an external device configured to direct the controller.

8. The system of claim 5, wherein the controller is configurable to operate between a sleep mode of operation and an awake mode of operation.

9. The system of claim 5, wherein the controller is at least partially controlled by input received into an application of a smart phone.

10. A method of preventing bruxism, comprising:
disposing an electrode within a user's mouth;
disposing a pressure sensor between the user's upper jaw and the user's lower jaw;
disposing an electrical stimulation generator within the user's mouth;
sensing a predetermined pressure; and
applying electrical stimulation to the user's mouth via the electrode in response to the sensed predetermined pressure.

11. The method of claim 10, wherein the electrical stimulation comprises a burst profile.

12. The method of claim 10, wherein the electrical stimulation comprises a continuous profile.

13. The method of claim 10, wherein the electrical stimulation comprises an amplitude modulated profile.

14. The method of claim 10, wherein the application of electrical stimulation is applied according to a first profile for a first period of time and subsequently according to a second profile in response to the first profile being unsuccessful in achieving a predetermined change in sensed pressure.

15. The method of claim 10, wherein the electrical stimulation comprises a low-frequency profile.

16. The method of claim 10, wherein the electrical stimulation comprises a high-frequency profile.

17. The method of claim 10, wherein the electrical stimulation comprises a high amplitude profile.

18. The method of claim 10, wherein the electrical stimulation comprises a low amplitude profile.

19. The method of claim 10, wherein the electrical stimulation comprises a short duration profile.

20. The method of claim 10, wherein the electrical stimulation comprises a long-duration profile.
